(19) United States
(12) Patent Application Publication
(10) Pub. No.: US 2007/0239033 A1
(43) Pub. Date: Oct. 11, 2007

(54) ARRANGEMENT, METHOD AND COMPUTER-ACCESSIBLE MEDIUM FOR IDENTIFYING CHARACTERISTICS OF AT LEAST A PORTION OF A BLOOD VESSEL CONTAINED WITHIN A TISSUE USING SPECTRAL DOMAIN LOW COHERENCE INTERFEROMETRY

(75) Inventors: Guillermo J. Tearney, Cambridge, MA (US); Brett Eugene Bouma, Quincy, MA (US)

Correspondence Address: DORSEY & WHITNEY LLP;INTELLECTUAL PROPERTY DEPARTMENT, 250 PARK AVENUE, NEW YORK, NY, 10177, US

(73) Assignee: The General Hospital Corporation, Boston, MA (US)

(21) Appl. No.: 11/687,112

(22) Filed: Mar. 16, 2007

Related U.S. Application Data
(60) Provisional application No. 60/783,600, filed on Mar. 17, 2006.

(51) Int. Cl. A61B 5/05; A61B 6/00

(52) U.S. Cl. 600/476; 600/473; 600/407

(57) ABSTRACT

An apparatus, method and computer-accessible medium for identifying characteristics of at least a portion of a blood vessel contained within a tissue can be provided. For example, it is possible to utilize a radiation source configured to provide a radiation to the tissue. In addition, a probe can be provided which may be adapted to receive the radiation returned from the tissue; Further, a system may be utilized which can be configured to process data relating to the tissue. The data can indicate whether the blood vessel is in a vicinity of an end portion of the probe.

### CROSS-REFERENCE TO RELATED APPLICATION(S)

This application is based upon and claims the benefit of priority from U.S. Patent Application Ser. No. 60/783,600 filed Mar. 17, 2006, the entire disclosure of which is incorporated herein by reference.

### STATEMENT REGARDING FEDERALLY SPONSORED RESEARCH

The invention was made with the U.S. Government support under Contract No. FA9550-04-1-0079 awarded by the Department of Defense. Thus, the U.S. Government has certain rights in the invention.

### FIELD OF THE INVENTION

The present invention relates to an apparatus and method for identifying tissue types using interferometric ranging during needle biopsy. More particularly, the present invention relates to an diagnosis system including a needle probe and algorithms for guiding vascular access.

### BACKGROUND OF THE INVENTION

A significant cause of inefficiency of intraoperative and biopsy procedures is the inability of a physician to identify tissue type by gross inspection. For example, head and neck surgeries, the inability to differentiate muscle, fat, lymph node, and parathyroid glands by gross inspection leads to unnecessary operative time, resulting in an increase in the cost of these procedures. Further, when not guided by an imaging modality, fine needle aspiration biopsies yield non-diagnostic tissue in 25% to 35% of cases. In medicine, there is a significant need for an inexpensive, portable, and efficient way for identifying tissue type.

The use of optical coherence tomography and confocal microscopy in needle probes has been previously described. These needle probes allow physicians to acquire images of tissue. However, these conventional needle probes have certain shortcomings. The methods used in these needle probes require imaging a single focused spot on a sample by scanning the spot in two dimensions in order to produce a two dimensional image of the subject. The scanning and imaging requirements of these known imaging needle probe systems require complex and expensive disposable components, as well as console components. Many components of existing imaging needle probes require complex and expensive construction making routine use of the needle probes a practical impossibility. Further, current imaging needle probes use complex and expensive custom syringes, which may not be sterilizable or disposable.

In the past, research has been performed to evaluate the use of low-coherence interferometry (“LCI”) imaging for tissue diagnosis. Optical coherence tomography (“OCT”) is LCI imaging that is performed by obtaining many axial scans while scanning a sample arm beam across a specimen, creating a two dimensional image. In order to perform LCI imaging, several strict requirements must be met by the conventional systems, including use of:
1. high speed reference arm delay scanning (at least 1,000 scans/second),
2. a high power broad bandwidth source (at least 5 mW),
3. a complex probe (must have at least one lens and a scanning mechanism),
4. an expensive data acquisition apparatus, and
5. an image display.

These requirements of the conventional systems dramatically increase the cost of OCT systems and OCT probes.

It may be beneficial to provide a low cost and accurate imaging system, process and needle biopsy probe having sufficient resolution that can be used by physicians with little additional training. It would also be desirable to have a needle biopsy probe that would use conventional syringe and needle combinations to avoid the high cost of developing and manufacturing custom barrels or needles. Such an exemplary system would also desirably be able to provide real time, or near real time, feedback regarding progress and location of the biopsy needle. Such a probe should also be able to identify various tissue types and interfaces and be able to alert a user when a target site has been reached or if an inappropriate tissue has been encountered. Interfaces are refractive index interfaces which occur when one tissue having optical refractive index is adjacent to another. The refractive index is unique to the molecular constituents of tissue and therefore interfaces occur throughout tissue. These refractive index interfaces may give rise to scattering which is the signal detected by LCI and OCT.

In addition to tissue identification, it may be beneficial to provide a device, method and computer-accessible medium capable of guiding vascular access. Obtaining vascular access is important in a number of medical scenarios. For example, in a trauma setting, obtaining vascular access can mean the difference between life and death for a patient in need of life-saving fluids and medications. This is especially the case in field-based medicine, where obtaining vascular access is often complicated by environment, stress, and lack of extra medical supplies. In addition, vascular access obtained incorrectly can lead to complications such as inflammation, thrombosis, and infection. Since manual palpation may often be the only cue for determining the optimal position of the needle prior to insertion into a blood vessel, obtaining vascular access is frequently difficult and time consuming. There exists a need for an active feedback in needle guidance for obtaining vascular access, particularly in the setting of military trauma.

Accordingly, it may be beneficial to address and/or overcome at least some of the deficiencies described herein above.

### OBJECTS AND SUMMARY OF THE INVENTION

The present invention generally provides devices, processes, software arrangements and storage media for identifying tissue types using interferometric ranging. The probe or disposable portion of the device uses a solitary single mode optical fiber, which is inexpensive and may fit into the lumen of a clinically available needle. The solitary single mode optical fiber can be between 125 μm and 250 μm in diameter.

According to one exemplary embodiment of the present invention, an apparatus, method and computer-accessible medium for identifying characteristics of at least a portion of a blood vessel contained within a tissue can be provided. For example, it is possible to use a radiation source configured to provide a radiation to the tissue. In addition, a probe can be provided which may be adapted to receive the radiation returned from the tissue; Further, a system may be utilized which can be configured to process data relating to the tissue. The data can indicate whether the blood vessel is in a vicinity of an end portion of the probe.

For example, according to a particular exemplary embodiment of the present invention, the probe can be configured to forward the radiation to the tissue from the radiation source. The probe and/or the system may be configured to generate axial scan data and/or transverse scan data based on the radiation. The system may be configured to identify characteristics of the tissue based on the axial scan data or the transverse scan data. The axial scan data may include data for backscattering, spectral properties, birefringence and/or Doppler shift. The transverse scan data may include data for the backscattering, spectral properties, birefringence and/or Doppler shift. The system can include an interferometer adapted to direct a portion of the radiation emitted by the radiation source into a sample arm and detecting radiation reflected from the tissue back through the sample arm. The system can be configured to identify characteristics of the tissue by processing the axial scan radiation to provide the characteristics of the tissue. The axial scan radiation may include the radiation received from the reference arm and the radiation received from the sample arm. The system can further compare the characteristics of the tissue with a database of normalized characteristics of a plurality of tissue types.

According to another exemplary embodiment of the present invention, the data may be unidimensional. The radiation source can be a light source configured to emit light. The light source can be a broad bandwidth light source and/or a swept wavelength optical source. The light source can deliver radiation to the tissue via an optical fiber disposed in an insertion device having a distal end at least partially disposed within the insertion device and a proximal end. The insertion device (which can be a barrel, a needle, and/or a stylet) may be configured to provide the distal end of the optical fiber adjacent to the tissue. The interferometer can direct another portion of the radiation into a reference arm.

In a still another exemplary embodiment of the present invention, the system can be configured to process the axial scan radiation by performing a standard deviation, average deviation, a special frequency information or a slope of the axial reflectivity profile relating to the axial scan radiation. The system can also be adapted to input data derived from the axial scan radiation into a statistical model to predict tissue type. The statistical model (e.g., a partial least squares or a principle component analysis model) can extract features from data derived from the axial scan radiation. Further, the system can be configured to identify the characteristics of the tissue by determining reflectance characteristics of the axial scan radiation using interferometric ranging, and compare the characteristics of the tissue with normalized reflectance characteristics of a plurality of types of tissue stored in a database. The type of interferometric ranging can be an optical time domain reflectometry, a spectral domain reflectometry and/or an optical frequency domain reflectometry.

According to yet another exemplary embodiment of the present invention, the data can relate to the axial scan radiation that is based on a spectral domain low-coherence interferometry and/or an optical frequency domain reflectrometry. The system can be configured to process the data to identify characteristics of the tissue. The data may also be based on a spectral domain low-coherence interferometry and/or an optical frequency domain reflectrometry. Further, the data can indicate a distance from the portion of the blood vessel to the end portion of the probe and/or at least one flow characteristic within the blood vessel.

For vascular access guidance , one exemplary method and arrangement for a guiding needle placement can integrate a sensor within the needle that could identify the proximity of a vessel during insertion, and specify when the tip of the needle has been successfully inserted into a vessel. One exemplary goal according to one exemplary embodiment of the present invention is to provide an optical sensor that can be incorporated within a needle and that can measure infrared reflectance, birefringence and flow as a function of distance from the tip. The sensor may comprise a passive optical fiber, resident within the needle bore, and a transceiver that can provide feedback cues to the operator.

Other features and advantages of the present invention will become apparent upon reading the following detailed description of embodiments of the invention, when taken in conjunction with the appended claims.

### BRIEF DESCRIPTION OF THE DRAWINGS

Further objects, features and advantages of the present invention will become apparent from the following detailed description taken in conjunction with the accompanying figures showing illustrative embodiments of the present invention, in which:

FIG. 1A is a graph of LCI reflectivity for muscle tissue;

FIG. 1B is a graph of LCI reflectivity for adipose tissue;

FIG. 2 is a schematic view of a tissue identification system according to one exemplary embodiment of the present invention;

FIGS. 3A-C are area schematic views of different fiber and probe designs according to one exemplary embodiment of the present invention;

FIG. 4 is a schematic view of an interferometric ranging probe in the lumen of a biopsy needle according to one exemplary embodiment of the present invention;

FIG. 5 is a schematic view of a syringe interferometric ranging probe with a single mode fiber inserted through the body of the syringe according to one exemplary embodiment of the present invention;

FIG. 6 is a schematic view of a syringe interferometric ranging probe with a single mode fiber inserted through the plunger of the syringe according to one exemplary embodiment of the present invention;

FIG. 7 is a schematic view of a syringe interferometric ranging probe with a single mode fiber inserted through an intermediate adapter between the syringe needle lock and the needle housing according to one exemplary embodiment of the present invention;

FIG. 8 is a schematic view of a syringe interferometric ranging probe with a single mode fiber inserted through an adapter between the syringe needle lock and the needle housing and includes a motion transducer according to one exemplary embodiment of the present invention;

FIG. 9 is a schematic view of a needle biopsy apparatus with an activation gun according to one exemplary embodiment of the present invention;

FIG. 10 is a schematic view of a cannula with an interferometric ranging probe in the body according to one exemplary embodiment of the present invention.

FIG. 11 is a schematic view of a cannula with an interferometric ranging probe in the lumen according to one exemplary embodiment of the present invention;

FIG. 12 is a schematic view of a cannula with an interferometric ranging probe in an electrocautery device according to one exemplary embodiment of the present invention;

FIG. 13A is a schematic view of a standard needle and housing;

FIG. 13B is a schematic view of a standard needle and a modified housing according to one exemplary embodiment of the present invention;

FIG. 14 is a schematic view of an interferometric ranging probe optical connector according to one exemplary embodiment of the present invention;

FIG. 15 is a schematic view of a biopsy probe with an associated feedback unit according to one exemplary embodiment of the present invention;

FIG. 16 is a schematic detail view of a gun and activation button according to one exemplary embodiment of the present invention;

FIG. 17 is a flow diagram of a method for tissue identification according to one exemplary embodiment of the present invention;

FIG. 18 is a schematic view of a system configuration according to one exemplary embodiment of the present invention;

FIG. 19A is a flow diagram of a signal processing sequence according to one exemplary embodiment of the present invention; FIG. 19B is a schematic diagram of an apparatus for identifying characteristics of at least one portion of a blood vessel; and

FIG. 20 is a flow diagram of a sequence according to one exemplary embodiment of the present invention for such identification of one or more portions of the blood vessel.

Throughout the figures, the same reference numerals and characters, unless otherwise stated, are used to denote like features, elements, components or portions of the illustrated embodiments. Moreover, while the subject invention will now be described in detail with reference to the figures, it is done so in connection with the illustrative embodiments. It is intended that changes and modifications can be made to the described embodiments without departing from the true scope and spirit of the subject invention as defined by the appended claims.

### DETAILED DESCRIPTION OF EXEMPLARY EMBODIMENTS

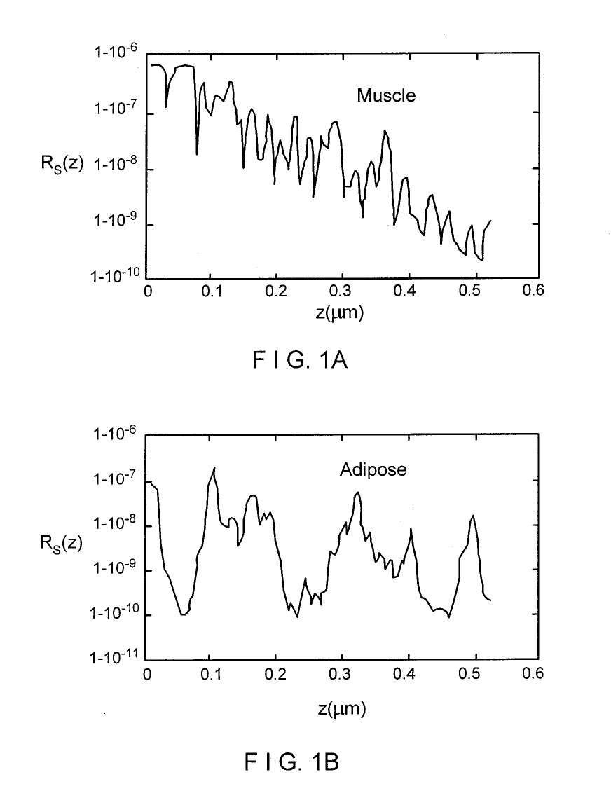

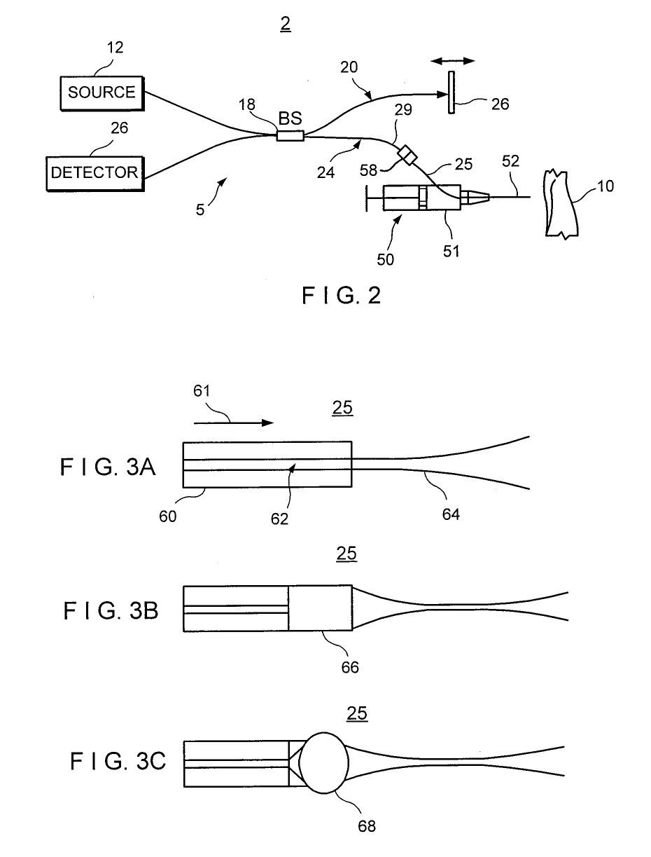

In accordance with the system of the present invention, FIG. 2 illustrates an tissue identification system 2 according to one embodiment of the present invention for tissue 10 identification using interferometric ranging. The tissue identification system 2 utilizes a one-dimensional data set in order to identify tissue. Unlike many prior art systems, which use two-dimensional data in order to acquire sufficient information to identify tissue, the tissue identification system 2 is able to identify tissue using a one-dimensional data set. Differences between two types of tissue may be understood from a one-dimensional data set. For example, FIG. 1 illustrates two graphs that represent a one-dimensional interferometric ranging axial scan of two different tissue types. As can be seen from these graphs, adipose tissue (shown in the bottom graph) has a significantly different axial reflectance profile as compared to the axial reflectance profile of muscle tissue (shown in the top graph). The tissue identification system 5 includes an imaging system 5 and a probe 50.

The imaging system 5 includes a light source 12, which is provided in an interferometer 14. The interferometer 14 can be a fiber optic interferometer 14. Also, while light is used in the disclosure herein as an exemplary embodiment of the light source 12, it should be understood that other appropriate electromagnetic radiation can be used, such as, microwave, radio frequency, x-ray, and the like. The interferometer 14 or other beam splitting device known to those skilled in the art may make use of circulators for increased sample arm power efficiency. The interferometer 14 includes a beam splitter 18, a reference arm 20, a sample arm 24, and a communications link to at least one detector 26. The light source 12 is connected to the interferometer 14 such that the light emitted from the light source 12 is transmitted to the beam splitter 18. The beam splitter 18 directs portion of the light emitted by the source 12 towards a reference arm 20, while the remainder of light is directed to a sample arm 24. The reference arm 20 includes a mechanism 26. The mechanism 26 produces a time dependent optical delay. In a certain embodiment, the mechanism 26 can be a movable reference reflector or mirror. The movable reference reflector or mirror can create a variable time delay suitable for a specific application.

An optical fiber 29, associated with the sample arm 24, is connected to an optical coupler 58. The optical coupler 58 is also connected to an optical fiber 25, which is inserted into the probe 50, as described below. The light signals returned from the sample arm 24 and the reference arm 20 are combined by the beam splitter 18 and reflectivity as a function of depth within the tissue sample 10 (e.g., see FIGS. 1A and 1B) is determined by measuring the interference between the two arms with at least one detector 26. Detection of a tissue birefringence (i.e., by splitting a ray into two parallel rays polarized perpendicularly) can be accomplished by using, e.g., two detectors 26, one for each polarization eigenstate. Depending on the type of interferometric ranging used, one to four detectors 26 may be employed.

In a certain embodiment, one of three types of interferometric ranging can be used: (i) optical time domain reflectrometry, (ii) spectral domain reflectrometry or (iii) optical frequency domain reflectrometry. It should be understood that additional alternate types of interferometric ranging could be used with the tissue identification system 2. If optical time domain reflectometry is utilized, the source 12 can be is a broad bandwidth light source, the interferometer 14 is needed, the reference arm 20 may be a low speed reference arm with delay scanning performing 20 to 50 scans per second, and the detector 26 can include one to four detectors. Optical time domain reflectometry is described in more detail by C. Youngquist et al., “Optical Coherence-Domain Reflectometry: A New Optical Evaluation Technique”, Opt. Lett., 12, 158-160 (1987), and K. Takada et al., “New Measurement System For Fault Location in Optical Waveguide Devices Based on an Interferometric Technique”, Appl. Opt. 26, 1603-1606 (1987), the entire disclosure of which are incorporated herein by reference. If spectral domain reflectometry is used, the source 12 is a broad bandwidth light source, the interferometer 14 is required, the detection arm includes a spectrometer, the detector 26 includes a single detector, and low coherence interferometry data is obtained by taking the Fourier transform of the measured spectrum. Spectral domain reflectometry is described in more detail by J. Deboer et al., “Improved Signal to Noise Ratio In Spectral Domain Compared With Time Domain Optical Coherence Tomography”, Optics Letters 2003, vol. 28, p. 2067-69; and Published Patent No. WO 03062802, entitled “Apparatus and Method for Ranging and Noise Reduction of Low Coherence Interferometry (LCI) and Optical Coherence Tomography (OCT) Signals by Parallel Detection of Spectral Bands”, to Deboer et al., the entire disclosure of both of which are incorporated herein. If optical frequency domain reflectometry is used, the source 12 is a swept wavelength optical source, the interferometer 14 is required, the detector 26 includes one to four detectors, and low coherence interferometry data is obtained by taking the Fourier transform of the measured spectrum. Optical frequency domain reflectometry is described in more detail by S. Yun et al., “High Speed Optical Frequency Domain Imaging”, Optics Express 2003, vol. 11, p. 2953-63, and C. Youngquist et al., “Optical Coherence-Domain Reflectometry: A New Optical Evaluation Technique, Opt. Lett., 12, 158-160 (1987), and K. Takada et al., “New Measurement System For Fault Location In Optical Waveguide Devices Based on an Interferometric Technique”, Appl. Opt. 26, 1603-1606 (1987), the entire disclosure of both of which are incorporated herein.

In an alternate embodiment of the present invention, the interferometer 14 is a Mach-Zehinder interferometer, a Michelson interferometer, a non-reciprocal or circular interferometer, a Sagnac interferometer, a Twyman-Green interferometer and the like. In another alternate embodiment of the present invention, the interferometer 14 is an interferometer as described in U.S. Provisional Application Ser. No. 60/514,769 filed Oct. 27, 2003, entitled “Apparatus and Method from Performing Optical Imaging Using Frequency-Domain Interferometry,” the disclosure of which is incorporated herein by reference in its entirety.

A probe 50 can include a biopsy device 51, which includes a needle 52 having a bore (not shown) associated with a syringe 54 through which the optical fiber 25 is introduced. The fiber 25 may be inserted into the probe 50 and in turn into the needle 52. The needle 52 and fiber 25 can be inserted percutaneously (or otherwise) toward the tissue 10 to be sampled. In other exemplary embodiments, the needle 52 can be a generic barrel, a specialized barrel, a needle, a stylet, and the like.

Referring now to FIG. 3, the fiber 25 includes a cladding 60 and a cleaved an optical fiber core 62, as shown in portion A of FIG. 3. When light signal is directed through the fiber 25 it forms a beam waist 64. The beam waist may be about 9 μm in diameter. Other lenses or optical elements may be attached to the fiber 25 to allow for focusing deeper into tissue, including a gradient index lens 66 (see portion B of FIG. 3), sometimes referred to as a GRIN lens, a ball lens 68 (see portion C of FIG. 3), a drum lens, a microlens, a tapered fiber end, a prism and the like. Alternatively, the fiber 25 may be angle cleaved or otherwise configured to produce an arbitrary pattern of electromagnetic radiation. In a certain embodiment, the cladding 60 has an outer diameter of 125 μm and the anoptical fiber core has an outer diameter of 9 μm.

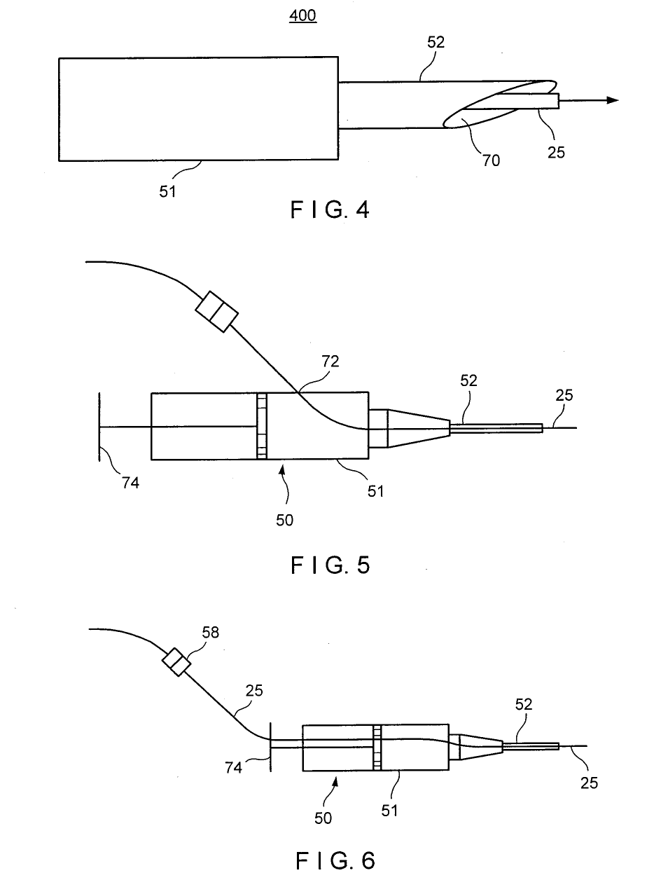

For needle biopsies that are traditionally performed using computerized tomography (CT), magnetic resonance imaging (MRI), or ultrasound guidance, the fiber 25 may be inserted into the biopsy needle 52 as shown in FIG. 4 and may be embedded within the needle biopsy device, or inserted through the lumen 70 of the needle 52. These types of procedures do not use fine needle aspiration. The lump or mass is not manually identifiable, but can only be identified through some other non-invasive imaging technique, such as CT or MRI. These and other guided needle biopsy procedures may use a larger and longer needle, while still utilizing the fiber 25 to assist in guiding the biopsy procedure.

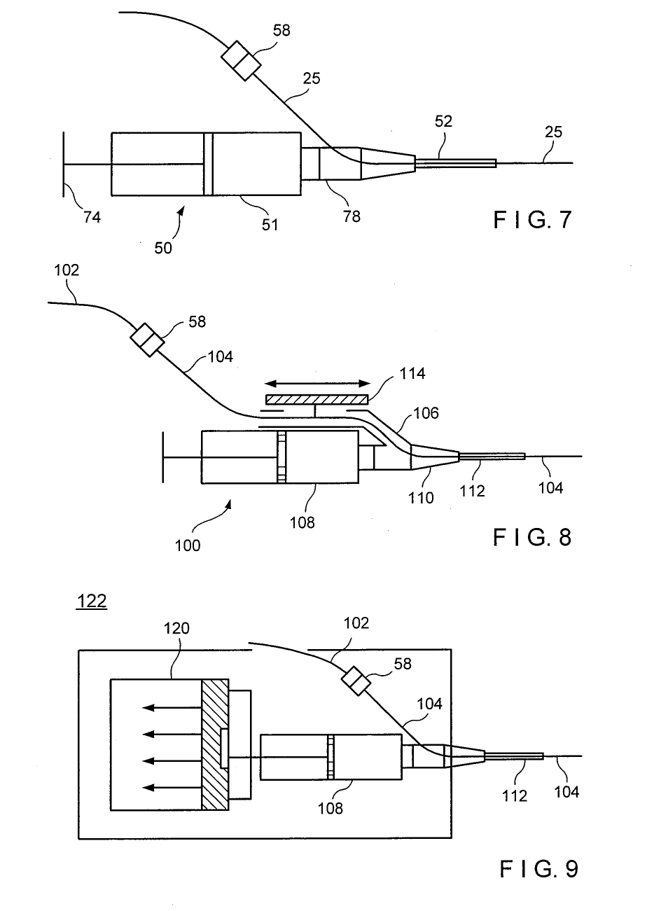

To insert the fiber 25 into the needle 52 of the probe 50 for fine needle aspiration, the fiber 25 may be inserted through an aperture 72, wherein FIG. 5 does not shown the aperture 72 in the body, in the body of the syringe 51 and then (i) into the needle 52 as shown in FIG. 5, (ii) through the plunger 74 of the syringe 51, and then provided into the needle 52 as shown in FIG. 6, (iii) through an intermediate piece 76 that is attached between the syringe 51 and the needle 52 as shown in FIG. 7, and/or by other insertion configurations. The probe 50 can be configured to allow suction for the aspiration of cells from the tissue 10, while allowing free movement of the fiber 25 at the tip of the needle 52.

In an exemplary embodiment, as shown in FIG. 8, the use of an intermediate coupler or holder between the syringe and the needle can be utilized. This would allow the use of standard needles and syringes. In this exemplary embodiment, a probe 100 utilizes the imaging system 5 as described above to identify tissue. The probe 100 includes an input fiber 102 attached to the imaging system 5 at one end, and to an optical connector 58 at the other end. The optical fiber 102 is connected to a single mode input fiber 104. The optical fiber 102 is inserted through an intermediate adapter 106, located between a syringe 108 and a needle lock 110. A needle 112 is attached to the needle lock 110. A motion transducer 114 may be used as a result of too little space between the outer surface of the fiber 104 and the inner bore surface of the needle 112. The motion transducer 114 generally allows the fiber 104 to be repositioned in order to allow aspiration of the tissue 10. The motion transducer 114 can be a manual motion transducer, an automated motion transducer, or the like. In another exemplary embodiment of the present invention, the needle lock 110 is a Luer lock.

FIG. 9 illustrates a tissue identification system 122 that includes the syringe 108 held within a device known in the art as a gun 120. This configuration allows for easy aspiration of the tissue 10 into the bore of the needle 112. Many of the components described above can also be incorporated into the tissue identification system 122 for easy access and convenience.

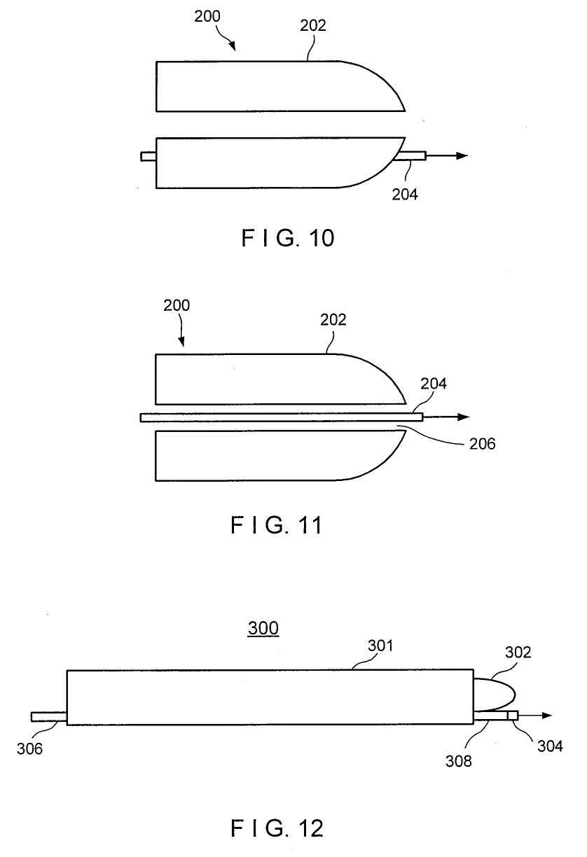

FIG. 10 illustrates an exemplary operation of placing a cannula 200 for IV access, pleural, perioneal taps, and the like according to a further embodiment of the present invention. The cannula 200 includes a guide catheter 202 and a fiber optic probe 204. The fiber optic probe 204 is provided within the guide catheter 202. Alternatively, the probe 204 may be inserted through the lumen 206 of the guide catheter 202 as shown in FIG. 11.

FIG. 12 illustrates an intra-operative exemplary embodiment 300 of a probe 306 according to still another embodiment of the present invention. The probe 306 is incorporated into an electrocautery device 301. An optical window 302 may be placed near the distal fiber tip 304 to protect the probe 306 against thermal damage by the cautery electrode 308. In yet another embodiment, the probe 306 may be incorporated into a scalpel, an independent hand-held device and the like instead of being incorporated into the electrocautery device 301. The optical window 302 can be made of sapphire.

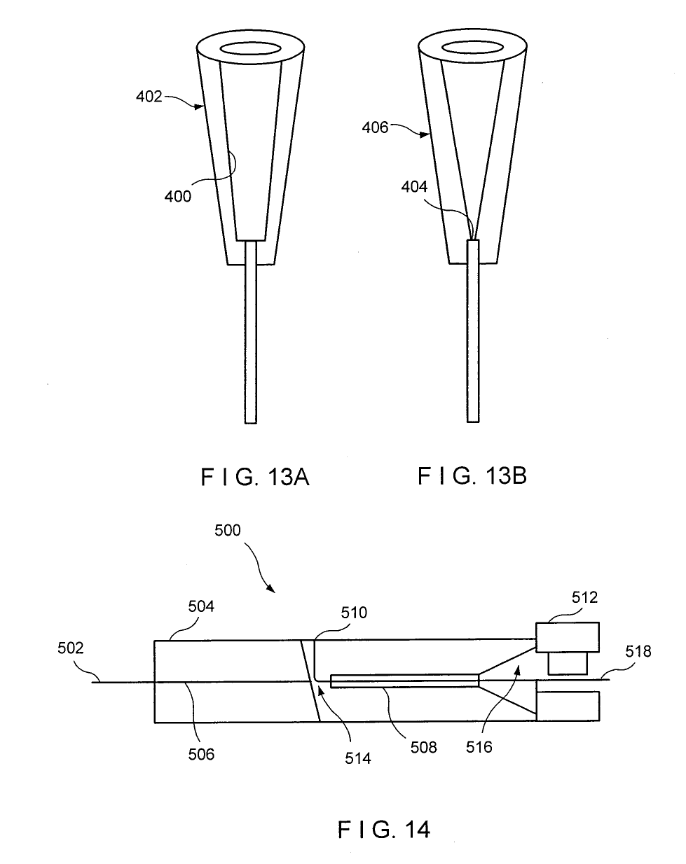

In order to allow for easy insertion of the fiber optic probe 25 into the needle 52, the internal lumen 400 of a standard needle housing 402, as shown in section A of FIG. 13, can be modified such that the internal lumen 404 of a modified needle 406 is tapered, as shown in FIG. 13B.

FIG. 14 illustrates an interferometric ranging probe optical connector 500, which is one side of the optical coupling 58, which can be used according to the present invention. The optical coupling 58, which connects the probe 50 to the imaging system 5, should be robust and simple to use. In another embodiment, the optical coupling 58 includes a bare fiber connector attached to the probe 50, which is relatively inexpensive, and the interferometric ranging probe optical connector 500 attached to the imaging system 5, which is relatively expensive. The use of a bare fiber connector attached to the probe 50 does not increase the cost of the probe 50. The more expensive portion of the optical coupling 58 is attached to the imaging system 5. The interferometric ranging probe optical connector 500 is constructed so as to engage with a bare fiber connector, such that a robust connection is made. The interferometric ranging probe optical connector 500 may include a cleaved (angle cleaved) fiber 502 (the proximate end of which is connected to the imaging system 5, not shown for the sake of clarity) inserted through a housing 504 having a ferrule 506 connected to a tapered v-groove 508. The tapered v-groove 508 is terminated by a fiber stop 510. The housing 504 has a taper 516 at one end through which a fiber 518 is inserted. The fiber 518 is inserted into the housing 504 via the taper 516 until it reaches the fiber stop 510. Once the fiber 518 comes to a stop, a clamp 512 holds the fiber 518 in place, away from the fiber-fiber interface, such that an air or fluid gap 514 is maintained. The fiber 518 is connected to the probe 50. In another embodiment, coupling gel may be used with flat cleaves to eliminate back-reflection from the gap 514.

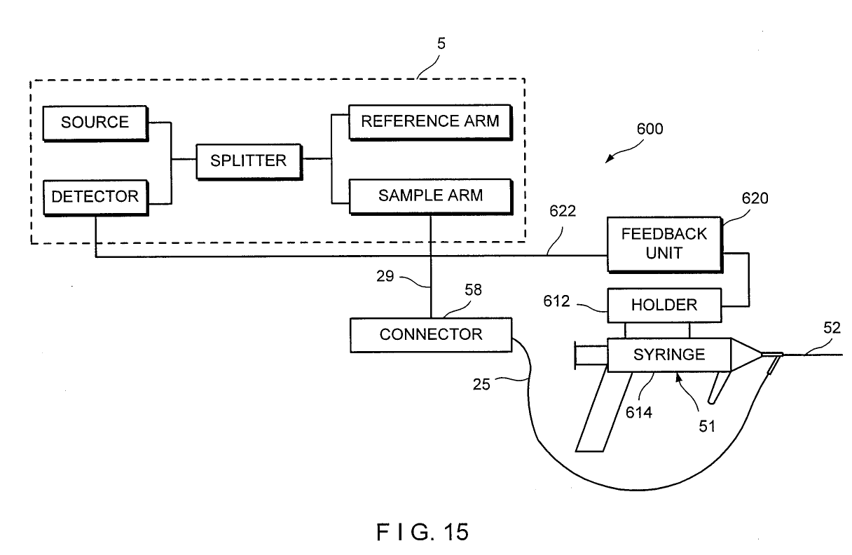

A number of optional mechanisms or apparatus configured to communicate specific information to a user regarding the tissue 10 being encountered by the tissue identification system 2 during a procedure may be used. FIG. 15 illustrates a schematic diagram of a system 600 with components of an imaging system 5 and the optical fiber 29 connected to the fiber 25 via the optical connector 58. The fiber 25 is operatively associated with a syringe 51 and passes through the bore of a needle 52. A holder 612 is associated with the syringe 51 by the syringe barrel 614. A feedback unit 620 can be associated with the holder 612 in any of several ways.

The holder 612 can be attached to the syringe 51. In an exemplary embodiment, the holder 612 is removably attachable to the syringe 51, such as, but not limited to, snap fit, removable adhesive, clamping, clipping or the like. By having the holder 612 be removably attachable to the syringe 51, the holder 612 and associated feedback unit 620 can be reused while the syringe 608 can be disposable, thereby enabling conventional syringes to be used and eliminating the need for a custom developed and expensive probes.

In another embodiment, the holder 612 is removably attachable to the syringe 51 using a gun or syringe holder. In another certain embodiment, the system 600 is integrally related to the gun (described above in relation to FIG. 9). In still another embodiment, the system 600 is embedded within the gun 634, which holds the feedback unit 620 and fiber 606 and improves the ability of the physician to aspirate tissue into the needle 610.

The feedback unit 620 provides information to the user of the system 600, including that the system 600 has detected tissue of a particular type. In another embodiment, the feedback unit 620 is a visual display, such as, LED, VGA, or other visual feedback system. With an LED display, the software algorithm and tissue identification determinations, as described herein below, can use an output signal to drive one or more LEDs, which can be actuated when the probe tip passes through or in proximity to differing tissue interface types (e.g., adipose versus muscle). As the tip contacts tissue of interest, such as a masticular lump, an LED light can change color or a different colored LED can be actuated to provide the physician feedback that the lump has been contacted and that the biopsy aspiration or other sampling can commence.

In still another embodiment of the present invention, the feedback unit 620 is an audible tone generator, which provides audio feedback as different tissue or other structures are detected by the system 600. In a further embodiment, the feedback unit 620 is a vibration generator. Each of the visual, audio and vibration feedback units provide simple and yet useful feedback to users of the system 600 to better target a biopsy probe in real time and with confidence. In yet another embodiment, the feedback unit 620 is a visual display screen that can be used to display a one or two-dimensional rolling plot image, comprising accumulated backscattered intensity as a function of z or depth within tissue, i.e. I_{Z}, over time to form an image. The visual display can be a conventional CRT display or an LCD display for providing more detailed or multimodal feedback. The visual display can be as small as or smaller than a conventional cell phone display or large to afford the user of the system 600 with a magnified view of the tissue 10.

The feedback unit 620 is communicatively coupled to the imaging system 602 by a physical cable connection 622 or via a wireless connection. The wireless connection can be a radio frequency (“RF”) connection, electromagnetic radiation signal, or the like. A wireless signal connection allows for reduced weight of the biopsy probe and fewer wires in the surgical site. A simple feedback system can be utilized so that the physician can operate the biopsy probe with one hand and have feedback proximate to the probe body so that the physician's concentration and visual focus does not leave the biopsy area.

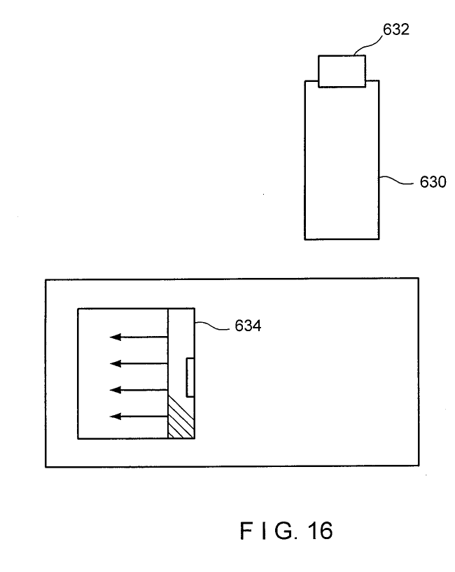

FIG. 16 illustrates a further embodiment of the feedback unit 620 including a display 630, a manually operated button or switch 632 and a gun 634. The switch 632 is operatively connected to the display 630. The actuation of the switch 632 causes the display 630 to show selection of standard biopsy procedures, such as, but not limited to, biopsy of breast tissue, liver tissue, spleen tissue, muscle tissue, lymph tissue, kidney tissue, prostate tissue and the like. Each of these biopsy procedures involves the probe 50 passing through relatively consistent types of layers, including skin, muscle, fascia, and the like, in a similar order for a given procedure. For example, for a lumbar puncture, the order of layers the probe 50 would encounter are skin fascia, vertebrae, muscle, fascia, disk, subdural space, epidural space, the spinal cord fluid area. Each of these tissues can produce a relatively consistent and determinable imaging signal peak which, when normalized over a substantial patient base by comparative image analysis and subtracting the curves of normalized data versus actual patient data, offers an accurate picture of what will be encountered during the biopsy procedure.

As the needle tip passes through each layer, the imaging system 600 detects the actual signal, and compares it to reference signals stored in a database. By taking an interferometric ranging scan of, for example, z (shown in FIG. 1) to obtain I(z), and taking the derivative dI/dz over time, a series of lines corresponding to the peaks of the sample may be obtained. The various consecutive peaks can be displayed by the feedback unit 620 to provide the user with accurate feedback of where the probe is and to assist the user in guiding the probe to the target area. The feedback unit can also incorporate an “anti-algorithm” to provide immediate feedback if the probe has wandered, overshot the target site or encountered a tissue type not expected to be detected during a particular procedure, such as, in the example of lumbar puncture, if the probe has passed the target area and hit a nerve. Such feedback can enable the physician to relocate the probe tip to the appropriate area.

The system and process according to the present invention is also able to determine when a target site has been reached. In order to determine when a target site has been reached, the system processes data from the reflected light to look for backscattering signatures that are indicative of a tissue type within the target site during a given procedure. Such processing consists of feature extraction and inserting these features into a model that predicts tissue type. This model can be a physical model, a chemometric model, or a combination of the two. A physical model generally predicts the scattering signal based on physical principles of light scattering. A chemometric model uses a training set and statistically extracts features using techniques such as Partial Least Squares (“PLS”) or Principle Component Analysis (“PCA”). Such model is developed based on known samples, and the new data can be tested using this model. It should also be understood that fringes may be acquired and processed to determine other tissue features including birefringence, Doppler flow, and spectral characteristics.

Tissue identification can be accomplished by visualizing the intensity, birefringence, Doppler, spectroscopic axial reflectivity profile and/or the like. Additionally, the frequency spectrum (Fourier transform of the intensity data) of the reflectivity scan will provide information relating to the spacing of the scattering structures in the tissue which relates to tissue structure. A more sophisticated analysis, including, but not limited to, variance analysis, one-dimensional texture discrimination (including fractal dimension, spatial gray level co-occurrence matrix parameters, Markovian distance, edge counting), power spectral analysis (including Fourier domain and time domain), n^{th} order histogram moment analysis, and temporal analysis of the reflectivity information (comparing one scan to another separated by a fixed time) using correlation techniques will provide information relating to the type of tissue observed. Other key quantitative metrics that may be used to characterize tissue types include, measurement of the backscattering coefficient, total attenuation coefficient, estimation of the anisotropy coefficient (particle size) from the onset of multiple scattering, particle shape and size from the detected spectrum using coherence-gated light scattering spectroscopy, and the like.

The following illustrative list of tissue characteristics may be found using the system and process of the present invention: adipose tissue, muscle tissue, collagen, nerve tissue, lymph node tissue, necrosis tissue, blood, glandular tissue and the like. Adipose tissue exhibits low absorbance at water peaks, high low spatial frequency components from the LCI intensity image and a high anisotropy coefficient. Muscle tissue exhibits high absorbance at water peaks, moderate birefringence, moderate anisotropy and decreased variance. Collagen exhibits very high birefringence. Nerve tissue exhibits moderate to high birefringence, high water absorbance and decreased power spectral density. Lymph node tissue exhibits a low anisotropy coefficient and a low temporal variance. Necrotic tissue exhibits high temporal variance of LCI signal, high attenuation coefficient, high water absorbance and low birefringence. Blood exhibits high Doppler shift, high water absorbance, high total attenuation coefficient, and high temporal variance. Glandular tissue exhibits moderate spatial frequency variance and low birefringence. It should be understood that the present invention contemplates the use of more than one analysis method, i.e., a multimodal system. This may provide enhanced detection and analysis of tissue types.

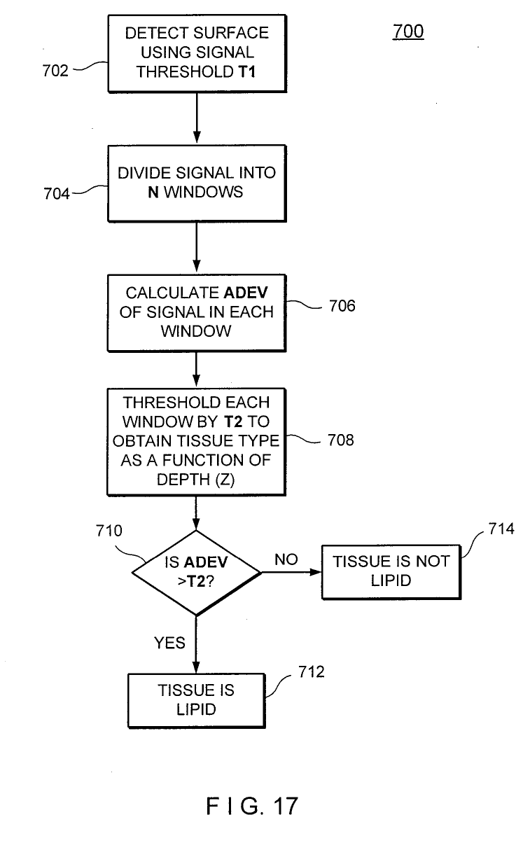

FIG. 17 illustrates a process 700 for differentiating fat tissue from fibrous tissue according to an exemplary embodiment of the present invention. The signal measured by the system 600 is an average of a number M axial scans. The system 600 detects the tissue sample surface using a signal threshold T1 at block 702. The detected signal is divided into N number of windows at block 704. Signal processing is conducted at block 706 to obtain a parameter derived from the interferometric ranging signal, such as the average deviation (ADEV) or standard deviation (STDEV) of the signal in each window (such as, but not limited to, the technique described in “Numerical Recipes in C”, Press, W. et al., Cambridge University Press, New York, N.Y. 1992, the entire disclosure of which is incorporated herein by reference) is calculated. Each window tested to determine if the threshold T2 is exceeded to obtain the tissue type as a finction of depth z at block 708. If, at block 710, the system 600 determines that ADEV (or STDEV) is greater than the threshold T2, the tissue is considered to be lipid, and the process 700 advances to block 712. Otherwise, the tissue is not likely to be lipid and the process 700 advances to block 714.

Applications of this technology can include tissue identification for the purpose of intraoperative guidance, needle biopsy guidance, fine needle aspiration, image guided biopsy, guiding placement of peripheral or central intravenous or intra-arterial catheters, and the like. Different methods of imaging can be used for different applications. These different applications include: guided biopsy; cell methodology; veni-arterio, pattern or Doppler recognition; lumbar, pattern; therapy guidance, pattern and optical methods; and the like. The probe 50 can also be used as a targeting and delivery device for therapeutics. The probe 50 can image the target area to make sure that needle injection of a therapeutic has reached and/or entered the target tissue or site by detecting the tissue type and interface change, i.e. a change in the refractive index of the tissue.

In order to determine whether the tissue is fibrous tissue or fat tissue, the process 700 utilizes standard image processing techniques to process data in order to differentiate fibrous (adipose) and fat tissue. Table 1, below, illustrates different measurements of fibrous and adipose tissue following the image processing of the data:

| TISSUE TYPE | SENS | SPECIFICITY |
|---|---|---|
| Fibrous | .95 | .98 |
| Adipose | .97 | .94 |
| Fibrous/Adipose | .96 | .84 |

In the table above, sensitivity is true positive, i.e. true positive+false negative, while specificity is true negative, i.e. true negative+false positive.

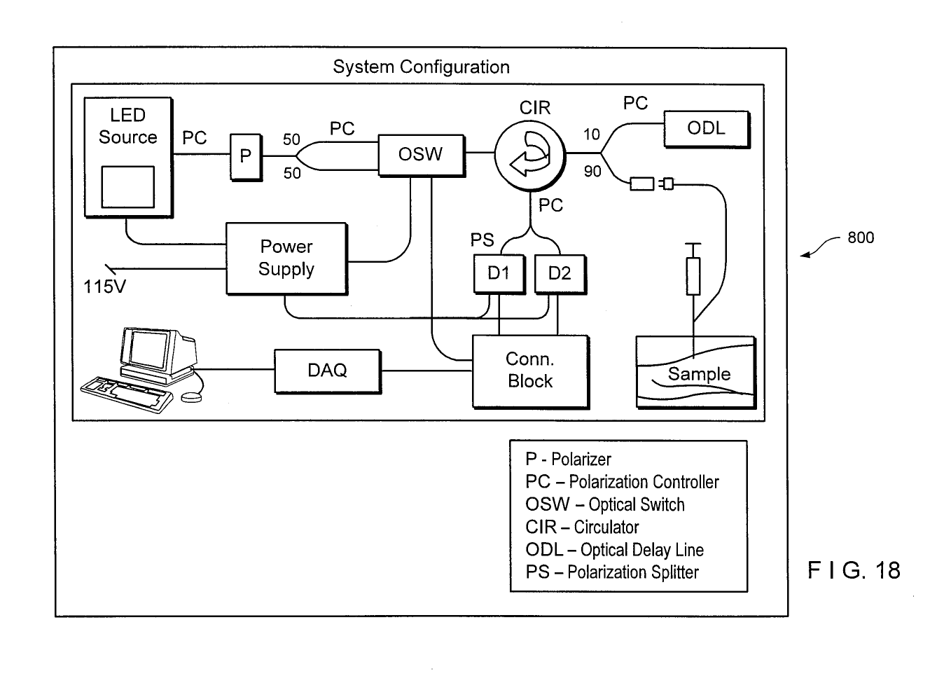

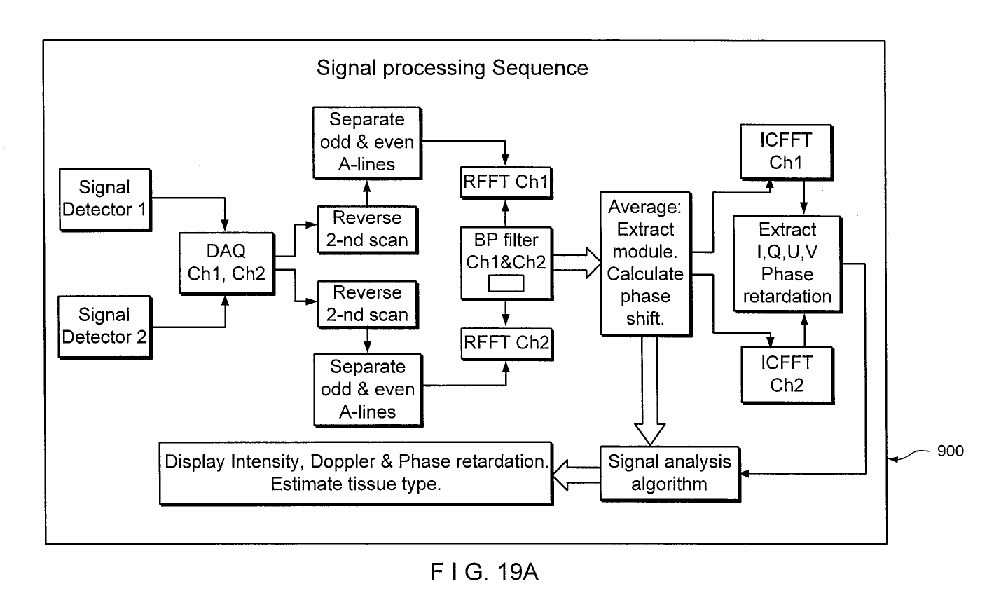

FIG. 18 illustrates an interferometric ranging diagnostic system 800 for identifying tissue according to the present invention. The system 800 uses a light source configured to emit light having a optical wavelength of 1.3 microns, 300 microwatts power and a 48 nm bandwidth. The light source allows the system 800 to interrogate tissue with a 15 micron resolution. The system 800 utilizes a low scanning frequency of the reference arm because building a coherent image is not the purpose of the system 800. Information can be gathered to identify the tissue through an average of several A-scans. The A-scan can be performed by one sweep of the reference arm, which corresponds to one depth scan. The system 800 processes and stores digital data, and the tissue type information is displayed on the feedback device in real time.

The LED source of the system 800 is a 300 microwatts SUPERLUM LED, which can be temperature and current controlled. The light is linearly polarized using a fiber optic polarizer P and sent to a beam splitter. The sample is interrogated with two orthogonally polarized states of the light in order to get birefringence information. The two orthogonally polarized states of the light are created by passing the light through the beam splitter, to two polarization controller PC paddles, one in each arm of the beam splitter. The two orthogonal polarization states are sent alternatively to the fiber optic circulator CIR, which directs the light to the fiber optic Michelson interferometer IF. The optical switch OSW is synchronized with the optical delay line ODL galvanometer, so that the polarization would change alternatively from one scan to the other. A very simple delay line, consisting in a retroreflector mounted to an lever driven by a galvanometer, was used to do the depth scanning. The probe attached to the sample arm of the interferometer IF includes a bare fiber introduced into a syringe needle. The backscattered light is coherently added to the light coming from the ODL and sent to the detectors D1, D2. A polarization splitter PS is used to select the two orthogonal states. The output signals of the detectors are preamplified and digitized using a NI DAQ card.

The system performs the digital acquisition, filtering, and averaging of the fringes, and provides the following information: (1) depth intensity at 15 microns resolution and spectral information, (2) birefringence information: computes stokes parameters-IQUV and extracts phase retardation, and (3) Doppler shift information.

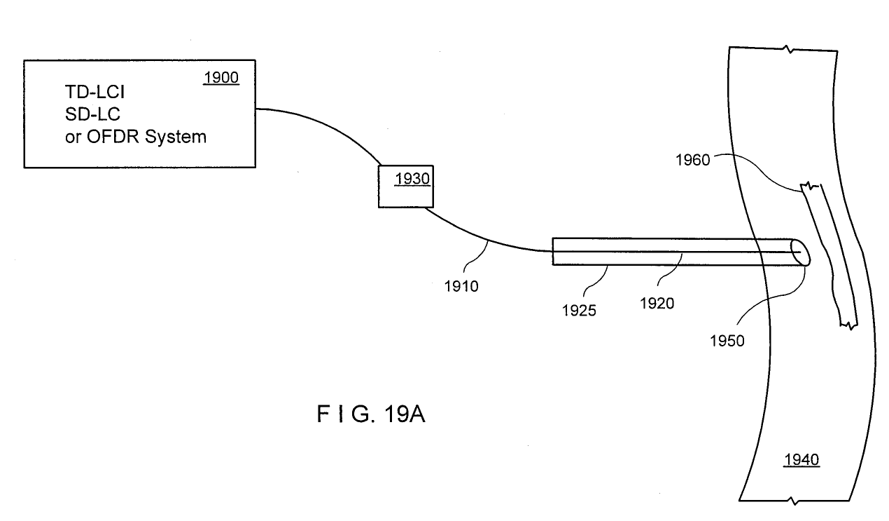

FIG. 19A illustrates an exemplary flow diagram 900 of the signal processing sequence according to an exemplary embodiment of the present invention. The simplest form of tissue identification is differentiation of two tissue types. The difference between two tissue types can be seen in FIG. 1, which shows the results of a feasibility study to distinguish cadaver fat from fibrous tissue. For example, adipose tissue has an appearance of multiple peaks separated by low interferometric ranging signal segments, whereas fibrous tissue has a lower degree of variance and decays exponentially.

Exemplary Vascular Access Guidance

FIG. 19B shows an exemplary arrangement according to the present invention for guiding vascular access. For example, one of criteria for a vessel differentiation may be determined by analyzing the axial SD-LCI or OFDR reflectance, birefringence, and Doppler flow profiles of cadaver arteries and veins, perfused by whole blood at a range of flow rates. According to one exemplary embodiment of the present invention, data can be collected as the needle is inserted through adjacent tissues to the vessel and after insertion within the vessels. According to another exemplary embodiment of the present invention, the SD-LCI or OFDR system may provide information regarding the distance of the needle tip with respect to the location of the blood vessel.

For example, a 850 nm or 1300 nm spectral-domain low-coherence interferometry (SD-LCI) 1900 or optical frequency domain reflectometry (OFDR) system 1900 can be used which is capable of measuring axial reflectivity (e.g., less than about 10 μm resolution), tissue birefringence, and Doppler flow. Light is transmitted from the SD-LCI or OFDR system through one or more optical fibers 1910 that is resident in the bore 1920 of the needle or stylet or incorporated as part of the needle within the wall of the needle 1925. Transverse information may be obtained by moving the optical fibers using a mechanical transduction unit associated with the optical fiber arrangement 1930.

When the needle is positioned at or inserted into the tissue 1940, the optical fiber arrangement 1910 may collect light returned from the tissue, and can transmit it to the exemplary SD-LCI or OFDR system 1900. A feedback signal can be provided to the operator, which can denote the existence/presence of a vessel 1960 in the field of view or the proximity or direction of the end portion of the probe and tip of the needle 1950 with respect to the vessel 1960. exemplary parameters including slope, standard deviation, and spatial frequency content may be extracted from the SD-LCI and or OFDR signal. Other quantitative metrics that may be used to characterize the tissue as vessel can include the backscattering coefficient, total attenuation coefficient, and anisotropy coefficient (e.g., related to particle size), which may be obtained from the SD-LCI signal. One exemplary criteria according to an exemplary embodiment of the present invention can be thereby provided for tissue differentiation, including epidermis, dermis, subcutaneous fat, muscle and fascia. Additional criteria may be provided to identify adventitia, media, intima and blood (static and/or flowing) that represent target features. Criteria may be obtained by obtaining information using cadaver or surgical specimens and comparing the information and data to gross and microscopic histopathology. Flow criteria may be identified by using blood flow phantoms in association with cadaver vessels.

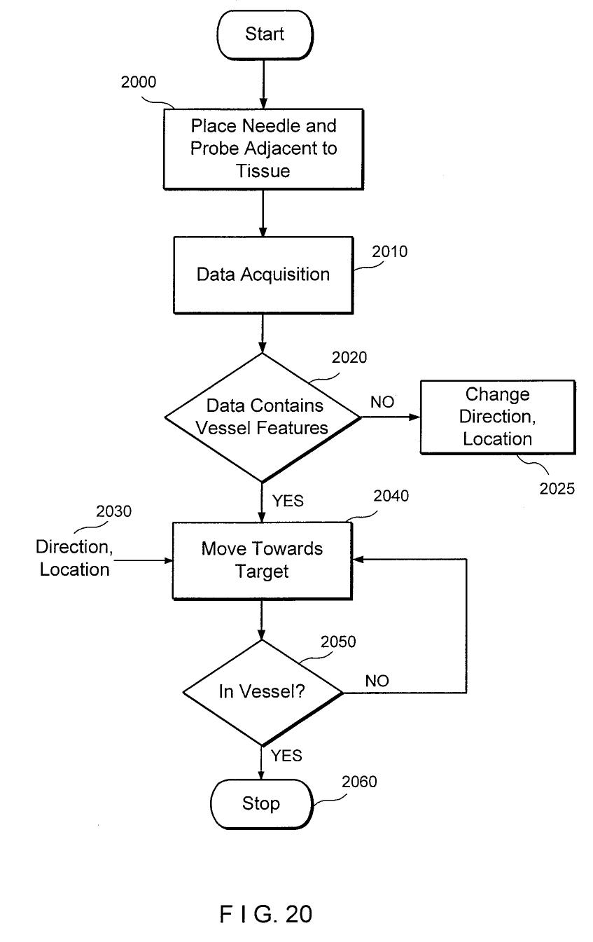

One exemplary embodiment of a method according to the present invention which can be used with a needle guidance system is shown in FIG. 20. For example, when the needle and probe is placed against the tissue (step 2000) or upon insertion of the needle or stylet into the patient, data acquisition commences (step 2010), and the exemplary system can look for target features that may represent the vessel (step 2020). If the target features are identified, the exemplary system can provide information regarding the location and direction of the vessel with respect to the end portion of the guidance probe (step 2030). The operator can then move the needle in a direction that brings the target features closer to the end portion of the probe (step 2040). If the target features are not identified, the operator may be instructed to move the needle until vessel features are identified (step 2025). When the probe is within the vessel (step 2050), the procedure can be considered as being complete (step 2060).

The foregoing merely illustrates the principles of the invention. Various modifications and alterations to the described embodiments will be apparent to those skilled in the art in view of the teachings herein. Indeed, the arrangements, systems and methods according to the exemplary embodiments of the present invention can be used with and/or implement any OCT system, OFDI system, SD-OCT system or other imaging systems, and for example with those described in International Patent Application PCT/US2004/029148, filed Sep. 8, 2004, U.S. patent application Ser. No. 11/266,779, filed Nov. 2, 2005, and U.S. patent application Ser. No. 10/501,276, filed Jul. 9, 2004, the disclosures of which are incorporated by reference herein in their entireties. It will thus be appreciated that those skilled in the art will be able to devise numerous systems, arrangements and methods which, although not explicitly shown or described herein, embody the principles of the invention and are thus within the spirit and scope of the present invention. In addition, to the extent that the prior art knowledge has not been explicitly incorporated by reference herein above, it is explicitly being incorporated herein in its entirety. All publications referenced herein above are incorporated herein by reference in their entireties.

What is claimed is:

1. An apparatus for identifying characteristics of at least a portion of a blood vessel contained within a tissue, comprising:
a radiation source configured to provide a radiation to the tissue;
a probe adapted to receive the radiation returned from the tissue; and
a system configured to process data relating to the tissue, wherein the data indicates whether the blood vessel is in a vicinity of an end portion of the probe.

2. The apparatus according to claim 1, wherein the probe is configured to forward the radiation to the tissue from the radiation source.

3. The apparatus according to claim 1, wherein at least one of the probe or the system are configured to generate at least one of axial scan data or transverse scan data based on the radiation.

4. The apparatus according to claim 4, wherein the system is configured to identify characteristics of the tissue based on at least one of the axial scan data or the transverse scan data.

5. The apparatus according to claim 3, wherein the axial scan data includes data for at least one of backscattering, spectral properties, birefringence or Doppler shift.

6. The apparatus according to claim 3, wherein the transverse scan data includes data for at least one of backscattering, spectral properties, birefringence or Doppler shift.

7. The apparatus according to claim 3,
wherein the system includes an interferometer adapted to direct a portion of the radiation emitted by the radiation source into a sample arm and detect radiation reflected from the tissue back through the sample arm, and
wherein the system is configured to identify characteristics of the tissue by processing the axial scan radiation to provide the characteristics of the tissue, the axial scan radiation including radiation received from the reference arm and radiation received from the sample arm, and compare the characteristics of the tissue with a database of normalized characteristics of a plurality of tissue types.

8. The apparatus according to claim 1, wherein the data is unidimensional.

9. The apparatus according to claim 1, wherein the radiation source is a light source configured to emit light.

10. The apparatus according to claim 9, wherein the light source is at least one of a broad bandwidth light source or a swept wavelength optical source.

11. The apparatus according to claim 9, wherein the light source delivers radiation to the tissue via an optical fiber disposed in an insertion device having a distal end at least partially disposed within the insertion device and a proximal end.

12. The apparatus according to claim 11, wherein the insertion device is configured to provide the distal end of the optical fiber adjacent to the tissue.

13. The apparatus according to claim 11, wherein the insertion device is one of a barrel, a needle or a stylet.

14. The apparatus according to claim 7, wherein the interferometer directs another portion of the radiation into a reference arm.

15. The apparatus according to claim 3, wherein the system is configured to process the axial scan radiation by performing at least one of standard deviation, average deviation, special frequency information and slope of the axial reflectivity profile relating to the axial scan radiation.

16. The apparatus according to claim 3, wherein the system is configured to input data derived from the axial scan radiation into a statistical model to predict tissue type.

17. The apparatus according to claim 16, wherein the statistical model extracts features from data derived from the axial scan radiation.

18. The apparatus according to claim 16, wherein the statistical model is at least one of a partial least squares model or a principle component analysis model.

19. The apparatus according to claim 3, wherein the system identifies the characteristics of the tissue by determining reflectance characteristics of the axial scan radiation using interferometric ranging, and comparing the characteristics of the tissue with normalized reflectance characteristics of a plurality of types of tissue stored in a database.

20. The apparatus according to claim 19, wherein the type of interferometric ranging is at least one of a optical time domain reflectometry, a spectral domain reflectometry or a optical frequency domain reflectometry.

21. The apparatus according to claim 3, wherein the data relates to the axial scan radiation that is based on at least one of a spectral domain low-coherence interferometry or an optical frequency domain reflectrometry, and the system is adapted to process the data to identify characteristics of the tissue.

22. The apparatus according to claim 1, wherein the data is based on at least one of a spectral domain low-coherence interferometry or an optical frequency domain reflectrometry.

23. The apparatus according to claim 1, wherein the data indicates a distance from the at least one portion of the blood vessel to the end portion of the probe.

23. The apparatus according to claim 1, wherein the data indicates at least one flow characteristic within the blood vessel.

24. A method for identifying characteristics of at least a portion of a blood vessel contained within a tissue, comprising:
providing a radiation to the tissue, and receive the radiation returned from the tissue; and
processing data relating to the tissue, wherein the data indicates whether the blood vessel is in a vicinity of an end portion of a probe which is configured to be inserted into the blood vessel.

25. A computer accessible medium for identifying characteristics of at least a portion of a blood vessel contained within a tissue providing thereon a software program, which, when executed by a processing arrangement, is operable to perform the procedures comprising:
performing a scan of the tissue using a radiation; and
processing data relating to the tissue, wherein the data indicates whether the blood vessel is in a vicinity of an end portion of a probe which is configured to be inserted into the blood vessel.
